(19) United States
(12) Patent Application Publication
(10) Pub. No.: US 2025/0345253 A1
(43) Pub. Date: Nov. 13, 2025

(54) CLEANING POWDER AND WHITENING ORAL CARE FORMULATIONS

(71) Applicant: Michael J. Florman, Garden Grove, CA (US)

(72) Inventor: Michael J. Florman, Garden Grove, CA (US)

(21) Appl. No.: 19/203,147

(22) Filed: May 8, 2025

Related U.S. Application Data
(60) Provisional application No. 63/643,929, filed on May 8, 2024.
(60) Provisional application No. 63/761,241, filed on Feb. 21, 2025.

(51) Int. Cl. A61K 8/22; A61K 8/02; A61K 8/19; A61K 8/20; A61K 8/66; A61K 8/92; A61Q 11/00

(52) U.S. Cl. CPC A61K 8/22; A61K 8/0241; A61K 8/19; A61K 8/20; A61K 8/66; A61K 8/922; A61Q 11/00

(57) ABSTRACT

A cleaning powder formulation for use in oral care includes a hydrogen peroxide powder source that is activated by mixing with water to create a hydrogen peroxide cleaning solution. The powder source ranges from about 40% to 98% of the total formulation.

### CROSS-REFERENCE TO RELATED APPLICATION

This application claims priority to U.S. Provisional Application No. 63/643,929, filed May 8, 2024, the disclosure of which is incorporated herein by reference in its entirety.

### FIELD

The present invention concerns a cleaning powder for oral care. The present invention also concerns a whitening oral care formulation.

### BACKGROUND

There are presently numerous oral care formulations on the market and cleaning solutions. Cleaning solutions can be used to clean dental equipment and appliances, like dentures, and also can be used to clean the mouth. Whitening formulations are also known. Since 1958, cyanuric acid and two chlorinated cyanurates, commonly referred to as dichlor (anhydrous sodium dichloroisocyanurate or sodium dichloroisocyanurate dihydrate) or trichlor (trichloroisocyanuric acid), have been added to outdoor swimming pools to stabilize chlorine residual by minimizing sunlight related degradation. Subsequently in July 2001, dichlor and trichlor were approved for routine drinking water treatment under the United States' Federal Insecticide, Fungicide, and Rodenticide Registration Act, and manufacturers have achieved NSF 60 certification for the addition of dichlor or trichlor to drinking water. Currently in the United States, dichlor or trichlor drinking water use may be approved by states, tribes, or territories.

Invitro testing demonstrated that dichloroisocyanurate whitened and cleaned stained test samples better than other cleaning formulations containing hydrogen peroxide. In fact, the testing demonstrated that dichloroisocyanurate outperformed both hydrogen peroxide cleaning solutions of up to 25% hydrogen peroxide and hydrogen peroxide salts (sodium perborate).

Tooth whitening has been performed for decades using hydrogen peroxide as the agent. H₂0₂ is an excellent agent for tooth whitening as the products released from the breakdown create an oxidation reaction, where one water molecule and a single oxygen molecule remain. The energy released during “oxidation” cleaves stain molecules into smaller molecules that do not have color. As a result, the surface where the stains existed no longer looks stained.

A summary of chlorinated cyanurate related chemical properties is shown below in Table 1.

TABLE 1

| | | | Sodium | |
|---|---|---|---|---|
| | | Sodium | Dichloroiso- | |
| | Cyanuric | Dichloroiso- | cyanurate | Trichloroiso- |
| Property | Acid | cyanurate | Dihydrate | cyanuric Acid |
| CAS | 108-80-5 | 2393-7809 | 51580-86-0 | 87-90-1 |
| Number | | | | |
| Chemical | C₃H₃N₃O₃ | C₃Cl₂N₃NₐO₃ | C₃Cl₂N₃NₐO₃•2H₂O | C₃Cl₃N₃O₃ |
| Formula | | | | |
| Molecular | 129.07 | 219.95 | 255.98 | 232.41 |
| Weight | | | | |
| % Available | 0 | 65 | 55 | 92 |
| Chlorine | | | | |
| (Theoretical) | | | | |
| % Cyanuric | 100 | 59 | 50 | 56 |
| Acid | | | | |
| (Theoretical) | | | | |
| pH, 1% | 4.8 ₐ | 6.0 _{b} | 6.0 _{b} | 2.8 _{b} |
| solution | (saturated | | | |
| unless | solution) | | | |
| otherwise | | | | |
| noted | | | | |
| Solubility | 2,010 ₐ | 24,000 _{b} | 28,000 _{b} | 1,400 _{b} |
| (mg/L H2O) | (25° C.) | (20° C.) | (20° C.) | (20° C.) |
| Total | Not | 0.81 | 0.69 | 0.76 |
| Chlorine (mg | Applicable | | | |
| Cl2/L) per | | | | |
| FIFRA Label | | | | |
| (Theoretical) | | | | |
| Total | Not | 0.73 | 0.63 | 0.46 |
| Cyanurate | Applicable | | | |
| (mg/L) per | | | | |
| FIFRA Label | | | | |
| (Theoretical) | | | | |

### SUMMARY

A cleaning powder formulation for use in oral care includes a hydrogen peroxide powder source that is activated by mixing with water to create a hydrogen peroxide cleaning solution, with the powder ranging from about 40% to about 98% of the total formulation.

The hydrogen peroxide powder source may be selected from the group of oxidizers that includes Hydrogen Peroxide-Releasing Powders, Sodium percarbonate (2Na₂CO₃·3H₂O₂), Sodium perborate (NaBO₃·nH₂O), Carbamide peroxide (urea hydrogen peroxide, CH₆N₂O₃), Calcium peroxide (CaO₂), Magnesium peroxide (MgO₂), Zinc peroxide (ZnO₂), Oxone, or triple salt potassium monopersulfate, potassium bisulfate, and potassium sulfate. The hydrogen peroxide powder source may range from about 40% to about 75% of the total formulation, or from about 40% to about 50% of the total formulation.

The formulation may further include one or more of a hydrogen peroxide catalyst or a non-metallic catalyst, with the catalyst designed to accelerate the breakdown of the hydrogen peroxide. The hydrogen peroxide catalyst may include one or more of transition metals including Iron (Fe²⁺/Fe³⁺), Manganese (Mn²⁺/Mn⁴⁺), Copper (Cu₊/Cu²⁺), Cobalt (Co²⁺/Co³⁺), Nickel (Ni²⁺), Silver (Ag⁺), Platinum (Pt), Palladium (Pd), or a transition metal compound and the non-metallic catalyst includes potassium iodide (KI) or sodium iodide.

The formulation may further include one or more of an enzyme, a ph modifier, a flavor derived from the oil of plants, and a sweetener. The enzyme is for breaking down and oxidizing the hydrogen peroxide and is an enzyme or a powder that includes the enzyme. The pH modifier may be one or more of sodium bicarbonate, sodium hydroxide, potassium hydroxide, and l-arginine.

The formulation may also include one or more of an oxidizer, a catalyst, a surfactant, and a metal chelator, a disinfectant, salts or minerals, and a phosphate.

In another embodiment, a whitening formulation for application to the teeth includes chlorine in the form of hypochlorous acid in its liquid state having a pH ranging between about 5 and about 11. The formulation may also include a thickening agent used to thicken the hypochlorous acid in its liquid state for application to the teeth.

The whitening formulation may also include a pH adjustment agent to adjust the levels of pH. The pH adjustment agent may be selected from the group of citric acid, acetic acid, sodium bicarbonate, or potassium hydroxide. The whitening formulation may also include a flavoring. The formulation may also include a chlorine masking agent, a scent, or a fragrance to mask any associated chlorine odor. The formulation may also include one or more artificial sweeteners. The artificial sweetener may be selected from the group comprising stevia, sucralose, xylitol, mannitol, sodium saccharin, and aspartame.

The formulation may also include one or more anticaries agents for decreasing tooth sensitivity and hydrogen peroxide to enhance tooth whitening action. The formulation can be placed directly on the teeth, positioned in a whitening tray and applied to the teeth, or positioned on a tooth whitening strip. The formulation may also include one or more of dental abrasives to aid in polishing the enamel of the teeth, metal scavenging agents, surfactants, humectants, vitamins, fluorides, enzymes, and emulsifiers.

### DETAILED DESCRIPTION

The present invention is directed to a cleaning powder formulation that is used for oral care and general cleaning purposes. The present invention is also directed to a teeth whitening formulation.

The cleaning powder formulation may be used to disinfect or clean dental appliances or can be used as a mouthwash. The cleaning powder contains one or more of a powdered oxygen releasing agent ingredient, a hydrogen peroxide catalyst, a non-metallic catalyst, an enzyme, and a ph modifier.

The powdered oxygen releasing agent ingredient may be selected from the group of oxidizers that include Hydrogen Peroxide-Releasing Powders, Sodium percarbonate (2Na₂CO₃·3H₂O₂), Sodium perborate (NaBO₃·nH₂O), Carbamide peroxide (urea hydrogen peroxide, CH₆N₂O₃), Calcium peroxide (CaO₂), Magnesium peroxide (MgO₂), Zinc peroxide (ZnO₂), Oxone, or triple salt potassium monopersulfate, potassium bisulfate, and potassium sulfate. The percentage of oxidizer may range from about 30% to about 80%. The percentage of oxidizer may alternatively range from about 40% to about 60%. Other ranges may alternatively be utilized.

The hydrogen peroxide catalyst may include transition metals such as Iron (Fe²⁺/Fe³⁺). Iron is especially important in the Fenton reaction (Fe²⁺+H₂O₂→Fe³⁺+⋅OH+OH⁻) where it produces reactive hydroxyl radicals. Another hydrogen peroxide catalyst is Manganese (Mn²⁺/Mn⁴⁺), which can catalyze H₂O₂ decomposition; Copper (Cu⁺/Cu²⁺), which can catalyze decomposition by cycling oxidation states; Cobalt (Co²⁺/Co³⁺), which strongly catalyzes peroxide breakdown; Nickel (Ni²⁺), which can have catalytic effects but is slower compared to iron or copper; Silver (Ag⁺), which can catalyze decomposition effectively, even at low concentrations; Platinum (Pt) and Palladium (Pd), both extremely strong catalysts that can cause rapid bubbling of O₂ gas when exposed to H₂O₂; or a transition metal compound such as manganese dioxide (MnO₂), silver, and platinum, among other known catalysts.

The non-metallic catalysts may include, for example, potassium iodide (KI) or sodium iodide.

The enzyme may be an enzyme or a powder that contains an enzyme within in it. Examples include catalases and peroxidases. Any other enzyme can also be used that catalyzes the breakdown and oxidation of hydrogen peroxide.

The pH modifier may include sodium bicarbonate, sodium hydroxide, potassium hydroxide, and l-arginine, as well as other known pH modifiers. pH modifiers, when hydrated will increase the pH of the formula, increasing the rate of breakdown of hydrogen peroxide.

Other oxidizers and ingredients may include potassium persulfate, and a halogenated isocyanurate, such as Dichloroisocyanuric acid (dichlor) or Trichloroisocyanuric acid (trichlor). A catalyst may be used that is selected from the transition metal category.

The mixture of powders can also include surfactants. Any of the categories of surfactants can be used alone or in combinations ranging from about 0.1% to about 4%, with about 1% to about 3% being a preferred range.

Anionic surfactants are known for their cleansing and foaming properties and are widely used in oral care and cosmetic products. These include Sodium lauryl sulfate (SLS), a popular foaming agent in toothpaste and cleansers; Sodium laureth sulfate (SLES), a milder foaming agent commonly used in facial washes and shampoos; Sodium lauroyl sarcosinate, a mild surfactant in toothpaste and baby care products; Sodium cocoyl isethionate, a gentle creamy cleanser often found in syndet bars; Disodium laureth sulfosuccinate, a very mild cleanser for sensitive skin formulations; Sodium methyl cocoyl taurate, a mild cleanser for sensitive or dry skin; Sodium myreth sulfate, similar to SLES but less irritating; Sodium lauryl sulfoacetate, used as a foaming agent in bath and oral products; Potassium laurate, occasionally used in toothpastes; and Sodium stearate, which also acts as a gelling agent in stick formulations.

Nonionic surfactants, which are known for their emulsifying and solubilizing capabilities, are also used in various cosmetic and oral care products. These include Polysorbates (Polysorbate 20, 60, 80), which solubilize oils and essential oils; Ceteareth-20/Ceteareth-12, emulsifiers in creams and conditioners; PEG-7 glyceryl cocoate, a mild cleanser and skin conditioning agent; Decyl glucoside, a plant-derived gentle surfactant found in baby care and oral care products; Coco glucoside, a mild cleanser commonly used in gentle products; Lauryl glucoside, another sugar-derived, mild foaming agent; Caprylyl/Capryl glucoside, a solubilizer for essential oils; and PEG-40 hydrogenated castor oil, a solubilizer in mouthwash and other oral care products.

Cationic surfactants, which are positively charged and typically used for conditioning and antimicrobial purposes, include Cetrimonium chloride, a conditioning agent in hair products; Behentrimonium chloride, commonly found in conditioners and emulsions; Cetylpyridinium chloride (CPC), an antimicrobial surfactant used in mouthwashes; and Stearalkonium chloride, a conditioner for hair and emulsions.

Amphoteric surfactants, which have both positive and negative charges, are mild and compatible with other surfactants. They include Cocamidopropyl betaine, a foam booster and surfactant used in oral products; Coco betaine, another foam booster and surfactant in cleansers; and Lauryl hydroxysultaine, which is used in foaming cleansers and oral rinses.

Natural surfactants are increasingly popular for clean beauty and oral care. These include Saponins (derived from soapwort, quillaja, or yucca), which act as natural foaming agents; Lecithin (from soy or sunflower), a natural emulsifier and surfactant; Soap bark extract (from Quillaja saponaria), a natural surfactant and foaming agent; Sucrose esters, biodegradable, food-grade emulsifiers; and Glyceryl oleate/Glyceryl stearate, used as mild surfactants in natural cosmetics.

Specialized surfactants, often dual-purpose, are used in oral care products. These include Sodium lauroyl lactylate, a mild surfactant in oral care; Poloxamers (Poloxamer 407), solubilizers commonly found in mouthwashes; PEG-8 and PEG-12, used as solubilizers and humectants in toothpastes; and Lauramide DEA/Cocamide DEA, surfactants used for thickening and foaming in older formulations.

The mixture of powders can also include a powdered metal chelator such as EDTA or others. The formulation may contain natural and or artificial flavors, in either a liquid form or powder form. The flavors can be essential oils or oils derived from plants, like mint. The percentage of essential oils or flavors or a combination of flavors may range from about 0.1% to about 5%, with about 1% to about 3% being more preferred.

Essential oils may also be included in the formulation and may include lavender oil, peppermint oil, tea tree oil, eucalyptus oil, lemon oil, sweet orange oil, frankincense oil, rosemary oil, bergamot oil, Roman chamomile oil, German chamomile oil, clove bud oil, clove leaf oil, ylang ylang oil, cinnamon bark oil, cinnamon leaf oil, cedarwood oil, sandalwood oil, grapefruit oil, geranium oil, patchouli oil, spearmint oil, lemongrass oil, juniper berry oil, myrrh oil, basil oil, thyme oil, oregano oil, marjoram oil, fennel oil, anise oil, cardamom oil, ginger oil, vetiver oil, cypress oil, fir needle oil, pine needle oil, black pepper oil, white pepper oil, carrot seed oil, helichrysum oil, neroli oil, rose otto oil, rose absolute oil, lime oil, mandarin oil, tangerine oil, petitgrain oil, palmarosa oil, niaouli oil, ravintsara oil, bay laurel oil, wintergreen oil, spikenard oil, melissa (lemon balm) oil, copaiba balsam oil, benzoin oil, elemi oil, galbanum oil, labdanum oil, hyssop oil, litsea cubeba (may chang) oil, tagetes oil, turmeric oil, sage oil, Spanish sage oil, savory oil, mugwort oil, angelica root oil, dill seed oil, coriander oil, cumin seed oil, juniper leaf oil, thuja oil, tea bush oil, violet leaf absolute, sweet birch oil, tarragon oil, cistus (rock rose) oil, gingergrass oil, hop oil, ho wood oil, manuka oil, kanuka oil, basil holy (tulsi) oil, black spruce oil, blue cypress oil, blue tansy oil, chamomile wild Moroccan oil, daisy oil, buchu oil, caraway oil, celery seed oil, costus root oil, davana oil, opopanax oil, parsley seed oil, perilla oil, rhododendron oil, rue oil, sea buckthorn oil, violet absolute, lemon eucalyptus oil, shiso oil, stylophorum oil, tonka bean absolute, wormwood oil, and zedoary oil.

The formulation may include an artificial or natural sweetener such as aspartame, acesulfame potassium (Ace-K), saccharin, sucralose, neotame, and advantame. Other sweeteners from natural sources include steviol glycosides (extracted from the Stevia rebaudiana plant), monk fruit extract (from Siraitia grosvenorii), and thaumatin, a sweet protein from the katemfe fruit. Additional synthetic or semi-synthetic sweeteners include cyclamate, alitame, dulcin, P-4000, and neohesperidin dihydrochalcone (NHDC). The percentage of artificial sweeteners included in the formulation may range from about 0.1% to about 5%, with about 1% to about 4% being more preferred.

The mixture of powders may include a powdered disinfectant, such as chlorhexidine gluconate or cetylpyridinium chloride.

The formulation may contain salts or minerals ranging from about 20% to about 60%, with about 30% to 50% being more preferred. Salts or minerals that may be added to the formulation include, but are not limited to, calcium carbonate, sodium bicarbonate, sodium carbonate, potassium carbonate, potassium bicarbonate, magnesium carbonate, ammonium carbonate, lithium carbonate, strontium carbonate, barium carbonate, disodium phosphate, monosodium phosphate, trisodium phosphate, potassium citrate, sodium citrate, chalk, limestone, dolomite, and marble. The filler may also include one of the following strong bases: calcium hydroxide, magnesium hydroxide, sodium hydroxide.

The formulation may contain a phosphate, such as sodium tripolyphosphate, sodium pyrophosphate, calcium glycerophosphate, disodium phosphate, monosodium phosphate, sodium metaphosphate, and potassium phosphate. A possible percentage of phosphates included in the formulation may range from about 0.5% to about 1.5%.

According to another aspect of the invention, a tooth whitening formulation is described below as utilizing a halogen element, specifically chlorine. The chlorine species is hypochlorous acid but can include other species of chlorine including Cl₂ at lower pHs to hypochlorate (OCl species) at higher pHs. The pH of the whitening solution is preferably between about 5 and about 11.

Hypochlorous acid can be produced through various common methods, such as Electrolysis of salt; pH adjustment of sodium hypochlorite (NaOCl); and dissolution of cyanuratic acid salts in water, such as dichloroisocyanuric acid and trichloroisocyanuric acid. The resulting hypochlorous acids produced by these methods is in a liquid form, with varying degrees of stability. To apply the hypochlorous acid to the teeth surfaces, it needs to be converted from a liquid to a gel that can stay in place on teeth surfaces. A thickening agent is used for this purpose.

Any thickening agent used in the manufacturing of cosmetics and/or oral care products can be used to thicken the hypochlorous acid in its liquid state. By using pH adjustment agents (acids or bases; most commonly acids), the solution is adjusted to the pH associated with the highest levels of hypochlorous (HOCl) and the lowest levels of hypochlorite (OCl). Such agents used to adjust pH can be an acid, not limited to citric acid, or acetic acid. pH increasing agents can be used, such as, but not limited to, sodium bicarbonate or potassium hydroxide.

Flavorings can be added to the formulation, such as, for example, peppermint, spearmint, cinnamon, bubblegum, lemon, or other flavors.

Chlorine masking agents and/or other scents and fragrances can be added to the formulation to mask any odors associated with the ingredients. Specifically, chlorine can produce a “pool smell” and masking agents can be used to mask that scent.

Artificial sweeteners such as (including but not limited to) stevia, sucralose, xylitol, mannitol, sodium saccharin, aspartame, and the like, may be utilized in the formulation. Anticaries agents can be added to decrease sensitivity that can accompany tooth whitening procedures. Dental tooth antisensitivity agents can be included in the formulation, such as potassium nitrate. Hydrogen peroxide can be added to the formulation to enhance the tooth whitening action. HOCl is an oxidizing agent and H₂O₂ is a reducing agent, producing Ox, HCl, and H₂O.

The formulation can be placed directly on the tooth in the dental office or placed in a dental tooth whitening tray and applied to the teeth by the dentist or by the patient at home. The agent can also be placed in a tooth whitening strip and applied to the teeth. The formulation can be combined with dental abrasives to aid in the enamel polishing step used during dental prophylaxis visits. It may also be used to polish the teeth during normal dental cleanings.

The formulation can be combined with metal scavenging agents (including but not limited to) etidronic acid, EDTA, and the like. The formulation can be combined with surfactants (including but not limited to) surfactants in the following categories: ionic, non-ionic, cationic, and amphoteric. The formulation can be combined with humectants (including but not limited to) propylene glycol or sorbital, glycerine, and the like. The formulation can be combined with thickening agents that include, but are not limited to, xanthan gum, carrageenan, hydroxyethylcellulose, hyaluronic acid, and the like.

The formulation can be combined with vitamins, such as vitamin A, E, B5, and K, among other vitamins. The formulation can be combined with fluorides that include, but are not limited to, sodium fluoride, stannous fluoride, acidulated phosphate fluoride, or sodium monofluorophosphate. The formulation can be combined with enzymes, such as alkaline phosphatase, catalase, amalayse, and peroxidase, among other known enzymes.

The whitening formulation can be combined with emulsifiers, including anionic emulsifiers, cationic emulsifiers, non-ionic emulsifiers.

In use, the formulation can be provided in different forms. A single dose package may include a syringe or cosmetic twist pen that is premixed and ready for application. A two-part system that needs to be mixed prior to application may have a Part A and a Part B. A three-part system may be provided that needs to have Part A mixed with Part B and then with Part C. The mixing of ingredients can be performed on a pad or in a small dappen dish. The mixing of the ingredients can include a combination of a syringe part and or a unit dose part, or a combination of the parts. The mixing of the ingredients described herein can be mixed directly on the tooth surface during application in one or more steps.

It should be noted that ingredients specified for use in connection with the Whitening formulation may also be used with the Cleaning Formulation. In addition, ingredients specified for use in connection with the Cleaning Formulation may also be used with the Whitening Formulation.

The term “substantially,” if used herein, is a term of estimation.

While various features are presented above, it should be understood that the features may be used singly or in any combination thereof. Further, it should be understood that variations and modifications may occur to those skilled in the art to which the claimed examples pertain. The examples described herein are exemplary. The disclosure may enable those skilled in the art to make and use alternative designs having alternative elements that likewise correspond to the elements recited in the claims. The intended scope may thus include other examples that do not differ or that insubstantially differ from the literal language of the claims. The scope of the disclosure is accordingly defined as set forth in the appended claims.

What has been described above includes examples of one or more embodiments. It is, of course, not possible to describe every conceivable modification and alteration of the above devices or methodologies for purposes of describing the aforementioned aspects, but one of ordinary skill in the art can recognize that many further modifications and permutations of various aspects are possible. Accordingly, the aspects described are intended to embrace all such alterations, modifications, and variations that fall within the spirit and scope of the appended claims. Furthermore, to the extent that the term “includes” is used in either the details description or the claims, such term is intended to be inclusive in a manner similar to the term “comprising” as “comprising” is interpreted when employed as a transitional word in a claim.

The term “consisting essentially,” if used herein, means the specified materials or steps and those that do not materially affect the basic and novel characteristics of the material or method. All percentages and averages are by weight unless the context indicates otherwise. If not specified above, the properties mentioned herein may be determined by applicable ASTM standards, or if an ASTM standard does not exist for the property, the most commonly used standard known by those of skill in the art may be used. The articles “a,” “an,” and “the,” should be interpreted to mean “one or more” unless the context indicates the contrary.

What is claimed is:

1. A cleaning powder formulation for use in oral care device cleaning comprising:
a hydrogen peroxide powder source that is activated by mixing with water to create a hydrogen peroxide cleaning solution, with the powder ranging from about 40% to about 98% of the total formulation.

2. The formulation of claim 1, wherein the hydrogen peroxide powder source is selected from the group of oxidizers that include Hydrogen Peroxide-Releasing Powders, Sodium percarbonate (2Na₂CO₃·3H₂O₂), Sodium perborate (NaBO₃·nH₂O), Carbamide peroxide (urea hydrogen peroxide, CH₆N₂O₃), Calcium peroxide (CaO₂), Magnesium peroxide (MgO₂), Zinc peroxide (ZnO₂), Oxone, or triple salt potassium monopersulfate, potassium bisulfate, and potassium sulfate.

3. The formulation of claim 1, further comprising a pH modifier in a range of about 40-60% and the hydrogen peroxide powder source being in a range of about 40-60% of the total formulation; and
optionally including an essential oil or an oil derived from plants, said oil being in a range of ½% to 5% of the total formulation.

4. The formulation of claim 3, wherein the pH modifier is selected from the group that includes sodium bicarbonate, sodium hydroxide, potassium hydroxide, and l-arginine; and
the oil is selected from the group that includes lavender oil, peppermint oil, tea tree oil, eucalyptus oil, lemon oil, sweet orange oil, frankincense oil, rosemary oil, bergamot oil, Roman chamomile oil, German chamomile oil, clove bud oil, clove leaf oil, ylang ylang oil, cinnamon bark oil, cinnamon leaf oil, cedarwood oil, sandalwood oil, grapefruit oil, geranium oil, patchouli oil, spearmint oil, lemongrass oil, juniper berry oil, myrrh oil, basil oil, thyme oil, oregano oil, marjoram oil, fennel oil, anise oil, cardamom oil, ginger oil, vetiver oil, cypress oil, fir needle oil, pine needle oil, black pepper oil, white pepper oil, carrot seed oil, helichrysum oil, neroli oil, rose otto oil, rose absolute oil, lime oil, mandarin oil, tangerine oil, petitgrain oil, palmarosa oil, niaouli oil, ravintsara oil, bay laurel oil, wintergreen oil, spikenard oil, melissa (lemon balm) oil, copaiba balsam oil, benzoin oil, elemi oil, galbanum oil, labdanum oil, hyssop oil, litsea cubeba (may chang) oil, tagetes oil, turmeric oil, sage oil, Spanish sage oil, savory oil, mugwort oil, angelica root oil, dill seed oil, coriander oil, cumin seed oil, juniper leaf oil, thuja oil, tea bush oil, violet leaf absolute, sweet birch oil, tarragon oil, cistus (rock rose) oil, gingergrass oil, hop oil, ho wood oil, manuka oil, kanuka oil, basil holy (tulsi) oil, black spruce oil, blue cypress oil, blue tansy oil, chamomile wild Moroccan oil, daisy oil, buchu oil, caraway oil, celery seed oil, costus root oil, davana oil, opopanax oil, parsley seed oil, perilla oil, rhododendron oil, rue oil, sea buckthorn oil, violet absolute, lemon eucalyptus oil, shiso oil, stylophorum oil, tonka bean absolute, wormwood oil, and zedoary oil.

5. The formulation of claim 4, further comprising a sweetener, wherein the sweetener permits a user to instantly use the cleaned device without rinsing.

6. The formulation of claim 2, wherein the hydrogen peroxide powder source ranges from about 40% to about 98% of the total formulation, or from about 40% to about 50% of the total formulation.

7. The formulation of claim 1, wherein the formulation further comprises one or more of a hydrogen peroxide catalyst or a non-metallic catalyst, with the catalyst designed to accelerate the breakdown of the hydrogen peroxide;
an enzyme;
a flavor; and
a sweetener.

8. The formulation of claim 1, wherein the hydrogen peroxide catalyst includes one or more of transition metals including Iron (Fe²⁺/Fe³⁺), Manganese (Mn²⁺/Mn⁴⁺), Copper (Cu⁺/Cu²⁺), Cobalt (Co²⁺/Co³⁺), Nickel (Ni²⁺), Silver (Ag⁺), Platinum (Pt), Palladium (Pd), or a transition metal compound and the non-metallic catalyst includes potassium iodide (KI) or sodium iodide; and
the enzyme is for breaking down and oxidizing the hydrogen peroxide and is either an enzyme or a powder that includes an enzyme.

9. The formulation of claim 6, further comprising one or more of an oxidizer, a catalyst, a surfactant, a metal chelator, a disinfectant, salts or minerals, and a phosphate.

10. A whitening formulation for application to the teeth comprising:
chlorine in the form of hypochlorous acid in its liquid state having a pH ranging between about 5 and about 11.

11. The formulation of claim 10, further comprising one or more of a thickening agent used to thicken the hypochlorous acid in its liquid state;
a pH adjustment agent to adjust the levels of pH;
a flavoring;
a chlorine masking agent, scent or fragrance to mask any associated chlorine scent;
an artificial sweetener;
an anticaries agent for decreasing tooth sensitivity; and
hydrogen peroxide to enhance tooth whitening action.

12. The formulation of claim 11, wherein the pH adjustment agent is selected from the group of citric acid, acetic acid, sodium bicarbonate, or potassium hydroxide; and
the artificial sweetener is selected from the group comprising stevia, sucralose, xylitol, mannitol, sodium saccharin, and aspartame.

13. The formulation of claim 10, wherein the formulation can be placed directly on the teeth, positioned in a whitening tray and applied to the teeth, or positioned on a tooth whitening strip.

14. The formulation of claim 10, further comprising one or more of dental abrasives to aid in polishing the enamel of the teeth, metal scavenging agents, surfactants, humectants, vitamins, fluorides, enzymes, and emulsifiers.

15. A whitening formulation for application to the teeth comprising:
chlorine in the form of sodium hydroxide with a pH ranging from 7 to 14.

16. The formulation of claim 15, wherein the pH of the sodium hydroxide is 14.

17. The formulation of claim 15, further comprising one or more of a thickening agent used to thicken the hypochlorous acid in its liquid state;
a pH adjustment agent to adjust the levels of pH;
a flavoring;
a chlorine masking agent, scent or fragrance to mask any associated chlorine scent;
an artificial sweetener;
an anticaries agent for decreasing tooth sensitivity; and
hydrogen peroxide to enhance tooth whitening action.

18. The formulation of claim 17, wherein the pH adjustment agent is selected from the group of citric acid, acetic acid, sodium bicarbonate, or potassium hydroxide; and
the artificial sweetener is selected from the group comprising stevia, sucralose, xylitol, mannitol, sodium saccharin, and aspartame.

19. The formulation of claim 15, wherein the formulation can be placed directly on the teeth, positioned in a whitening tray and applied to the teeth, or positioned on a tooth whitening strip.

20. The formulation of claim 15, further comprising one or more of dental abrasives to aid in polishing the enamel of the teeth, metal scavenging agents, surfactants, humectants, vitamins, fluorides, enzymes, and emulsifiers.
